(19) United States
(12) Patent Application Publication
(10) Pub. No.: US 2003/0163182 A1
(43) Pub. Date: Aug. 28, 2003

(54) Ice pack

(76) Inventor: Charles P. Hickey, Monroe, WI (US)

Correspondence Address: Stephen Michael Patton, NILLES & NILLES, S.C., Firstar Center, Suite 2000, 777 East Wisconsin Avenue, Milwaukee, WI, 53202-5345, US

(21) Appl. No.: 10/082,676

(22) Filed: Feb. 25, 2002

(51) Int. Cl. A61F 7/00

(52) U.S. Cl. 607/108; 607/112; 607/114

(57) ABSTRACT

An ice pack is provided having an inner waterproof container for holding ice and an outer fabric pouch for holding the inner container. Four straps having a dense pattern or pile of loops extend from the pouch. The loops on the strap can be mechanically coupled with a strip of corresponding hook fastener material having a dense pattern or pile of hooks that is fixed to the outer pouch.

### FIELD OF THE INVENTION

The invention relates to self-contained chilling devices such as ice packs.

### BACKGROUND OF THE INVENTION

Ice packs have been used for many centuries to chill human skin to treat minor ailments such as bruises and sprains. These devices are typically in the form of a soft, flexible container with one or two walls having a waterproof opening for containing the melted ice water inside.

One problem with these devices has been the need to tie or otherwise mechanically engage these devices, such as with zippers , buttons, ties or clasps. All of these devices suffer from a serious drawback in that they require considerable mechanical manipulation and, often, such as in the case of buttons, snaps or zippers, cannot be adjusted for length.

What is needed therefore is an ice pack having straps that can be easily, removably and replaceable attached to form loops of varying lengths.

### BRIEF DESCRIPTION OF THE DRAWINGS

FIG. 1 is a plan view of an ice pack in accordance with the present invention;

FIG. 2 is a cross-sectional view of the ice pack of FIG. 1 showing the several layers of material forming the ice pack;

FIG. 3 is a perspective view of the ice pack of FIGS. 1 and 2 with the zipper open and the sides distended to admit ice particles;

FIG. 4 is a perspective view of the ice pack of the foregoing FIGURES as it is applied to an arm of the human body with the ice pack straps wrapped around the arm and tied to one another;

FIG. 5 is a perspective view of the ice pack of the foregoing FIGURES as it is applied to an arm of the human body with the ice pack straps wrapped around the arm and fixed to the outer fabric pouch of the ice pack by mechanical engagement of loops on the straps with corresponding hooks on the outer fabric pouch;

FIG. 6 is a perspective view of the ice pack of the foregoing FIGURES as it is applied to a leg of the human body and fixed thereto by mechanical engagement of loops on the straps with hooks on the outer fabric envelope;

FIG. 7 is a perspective view of the ice pack of the foregoing FIGURES as it is applied to a human torso by engaging the loops on pairs of straps with hooks on two cinches;

FIG. 8 is a perspective view of the cinch of FIG. 7 in the form of a planar sheet having an aperture for receiving the straps and a dense pattern of hooks on at least one outer surface thereof;

FIG. 9 is a perspective view of the cinch of FIGS. 7 and 8 as it appears when engaged to the traps of the ice pack by mechanical engagement of the hooks on the cinch with the loops on two straps; and

FIG. 10 is a perspective view of the cinch of FIGS. 7 and 8 as it appears when it is engaged to two straps extending through the aperture of the cinch for strain relief and engaged with the hooks on the cinch.

### DETAILED DESCRIPTION OF THE PREFERRED EMBODIMENT

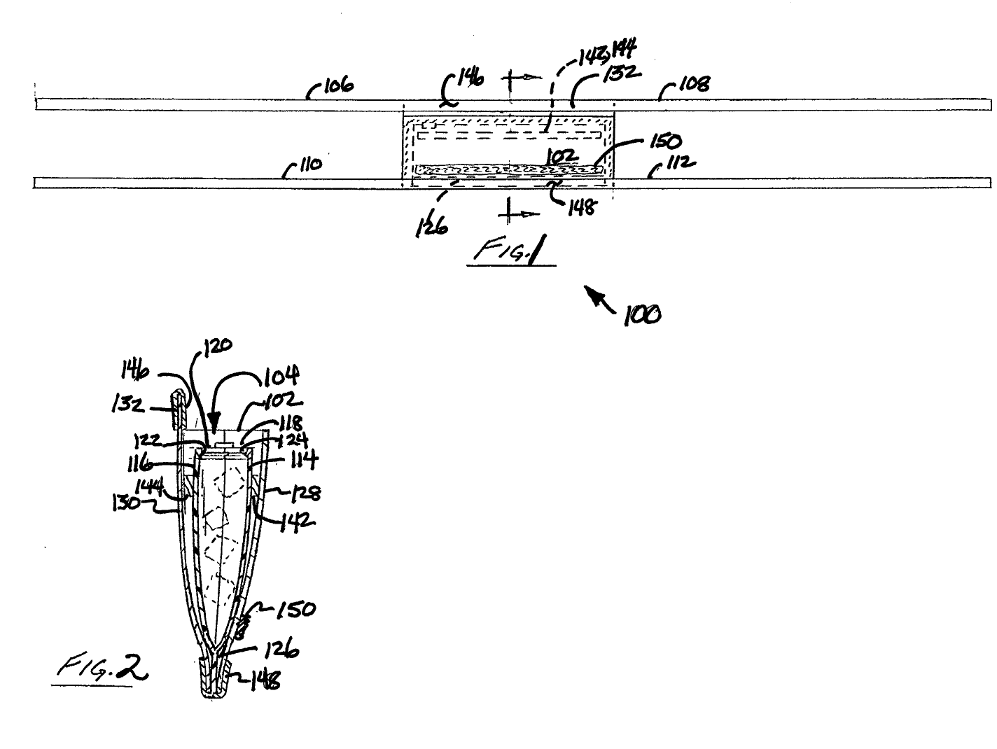

Referring to FIG. 1, the ice pack can be seen in plan view. When reference is made in this application to “top”, “bottom”, “left”, “right”, “sides”, “upper”, “lower”, or “lateral” in this application it refers to the orientation of the ice pack as it appears in FIG. 1. The term “front” refers to the surfaces facing the observer in FIG. 1. The term “back” refers to the surfaces facing away from the observer in FIG. 1.

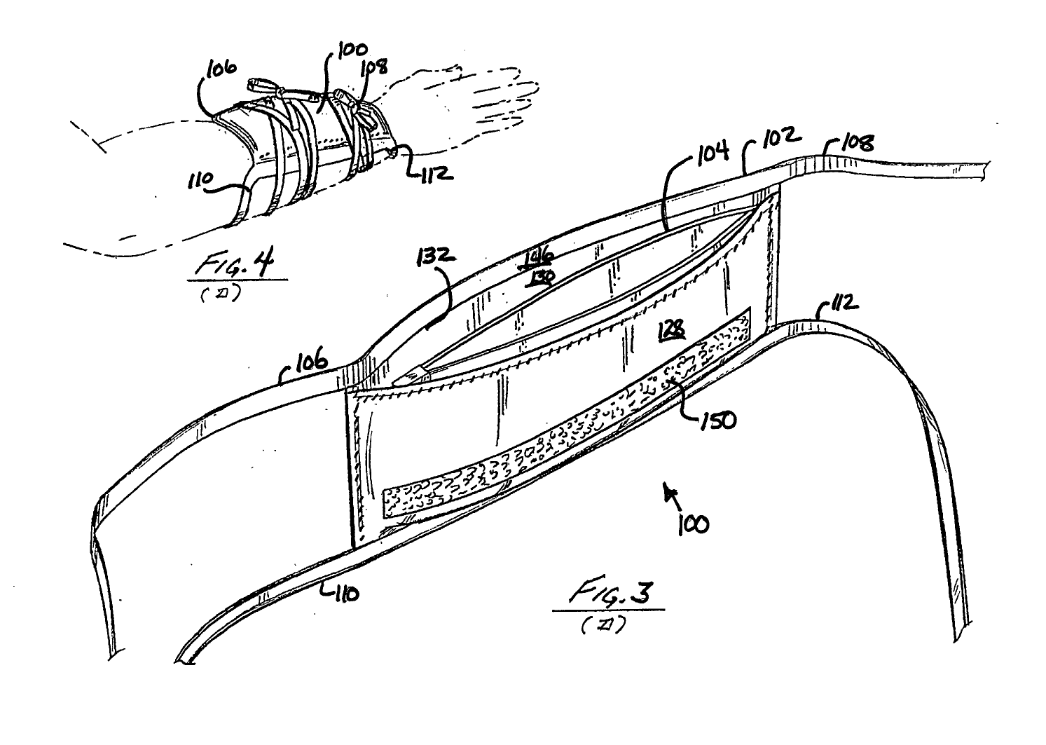

Referring to FIGS. 1-3, the ice pack 100 includes an outer fabric pouch 102 that surrounds and supports an inner waterproof container 104. Ice pack 100 also includes four straps 106, 108, 110, 112 that are fixed to the outer fabric pouch 102, extending laterally from each comer of pouch 102.

The inner waterproof container 104 is formed of two planar sheets, a front sheet 114 and a back sheet 116. These sheets are joined at their bottom edges and two side edges to form a pouch and to define a mouth 118 that can be opened or closed by means of a zipper 120.

The zipper is preferably in the form of two opposing interlocking strips 122, 124 that have ribs extending inwardly toward one another. The ribs extend laterally across the mouth of container 104 and interlock to provide a watertight seal at the mouth of container 104. This seal couples the two top edges of the planar sheets 114 and 116 together.

The planar sheets 114 and 116 are preferably rectangular in shape to define a generally rectangular container 104 wherein the lateral sides of the container are generally parallel and the zipper is parallel to the bottom of the container.

Container 104 preferably includes a short flap 126 that extends downward from the container where the two planar sheets are joined together to form the bottom edge. This flap or skirt, when provided, is used to anchor the bottom of the container to the outer fabric pouch.

The outer fabric pouch 102 encloses the inner container 104. It is comprised primarily of two opposed planar sheets: a front sheet 128 and a back sheet 130, that preferably made of non-woven spun lace thermoplastic materials, such as Sontara and Hydrophillic 8441, which are both trade names of Dupont Chemical Company. The materials are preferably comprised of polyester, rayon, nylon or a combination of the three. The sheets have opposed side edges that are extend vertically, and top and bottom edges that extend horizontally. This arrangement defines four comers: top left, top right, bottom left and bottom right.

The sheets are coupled along their lateral edges and along their bottom edges to form a pouch with an open mouth that is sized to receive container 104. The edges may be formed simultaneously and integrally with the planar sheets themselves, the sheets may be formed individually and fused at their edges, or the edges may be created by folding a single sheet and fusing its other edges.

Each of the two sheets 128 and 130, in turn, is preferably comprised of two sheets of material. Thus, sheets 128 and 130 preferably have an inner layer (i.e. the inner surface facing the inner container 104) that is formed of a planar sheet of hydrophillic material, such as Hydrophillic 8441 (trademark of Dupont Chem. Co.) and an outer layer (i.e. the layer forming the outer surface of pouch 102) that is formed of a non-woven fabric, such as Sontara (trademark of Dupont Chem. Co.).

Alternatively, the outer layer of sheets 128 and 130 may be a knit polyester fabric which is preferably fused to the inner hydrophillic layer to create the bi-layer fabric from which sheets 128 and 130 are formed.

In the preferred embodiment, the sheets 128 and 130 are individually formed and are fused at their respective bottom and side edges to form the pouch. The fusing is preferably heat fusing, and preferably employs an ultrasonic platen or roll to generate the heat of fusion.

Flap 126 extends downward from the bottom of container 104 and between sheets 128 and 130. The bottom of flap 126 is fixed to the bottom of fabric pouch 102, thus anchoring the bottom of container 104 (from which the flap extends) to the bottom of pouch 102. This reduces the chance that container 104 will be pulled inside out when the ice or water in container 104 is emptied.

Flap 126 preferably extends from the bottom edge of container 104 over the entire horizontal length of the bottom edge of container 104. While this is the preferred embodiment, a smaller width may also be used to anchor the bottom of the container 104 to the bottom of the pouch 102.

The top of back sheet 130 extends above the top of front sheet 128 in pouch 102. This extension 132 provides a mounting surface for straps 106 and 108 which extend laterally to the left and right from the upper left and upper right comers of outer pouch 102. In this manner, the mouth of pouch 102 remains accessible when the pouch is attached to the human body by straps 106 and 108.

There are fours straps that extend laterally from the outer pouch 102. These straps include straps 106 and 108, which are fixed to the upper edge of rear sheet 130 and the upper left and upper right respective comers of pouch 102. It also includes straps 110 and 112, which extend laterally away from the lower left and lower right respective comers of pouch 102.

At least a portion of each of these four straps comprises a loop fabric that includes a substrate and a dense pattern or pile of fabric loops extending therefrom. This loop fabric is preferably a knitted thermoplastic fabric such as a polyester knit that can be heat fused to front and back sheets 128 and 130 to attach the straps thereto. A preferred material is “Light Knitted Loop” by Dunlap Industries, Inc. of Chattanooga, Tenn., which has a brushed loop knap or pile on one side. In this embodiment, the loop material is folded over with the non-brushed portion facing inside and the brushed portion facing outside in order to form the strap, as described herein. The material is preferably nylon, although polyester or rayon fabrics (or any combination thereof) are also acceptable.. Alternatively, the loop fabric may be a more robust material, such as any of the loop fabrics sold under the trade name “Velcro®” or similar materials.

In the preferred embodiment, the straps are comprised of a single layer of knitted fabric that is folded over upon itself to form a double thickness. This folded-over material may be mechanically sewn together or, more preferably, may be thermally fused over its length, such as by using a heated or ultrasonic roller.

Straps made of knit fabric or other relatively flexible materials preferably have a width of between 0.375 and 1.5 inches, a width sufficient to permit them to be bent upward (straps 106 and 108) or downward (straps 110 and 112) 90 degrees for attaching the ice pack to a portion of a body such as an arm or leg, as shown in FIGS. 4-7.

Top straps 106, 108 are preferably formed from one continuous web of material that extends from the leftmost distal end of strap 106 to the rightmost distal end of strap 108. A central portion 146 of this web, best shown in FIG. 2, is folded over to enclose the top edge of rear sheet 130 over its entire top width. Central portion 146 is bonded to the top of rear sheet 130 preferably by heat fusion, such as by a heated or ultrasonic platen or roller.

In a similar fashion, the bottom straps 110, 112 are also preferably formed from one continuous web of material that extends from the leftmost distal end of strap 110 to the rightmost distal end of strap 112. A central portion 148 of this web, best shown in FIG. 2, is folded over to enclose the bottom edges of both front sheet 128 and rear sheet 130 over their entire bottom width. Central portion 148 is bonded to the bottom of edges of sheets 128 and 130 preferably by heat fusion, such as by a heated or ultrasonic platen or roller.

Ice pack 100 includes two layers of adhesive disposed on either side of container 104 adjacent to the mouth of container 104. A first adhesive layer 142 is disposed between and adhesively bonded to the inner surface of sheet 128 and the outer surface of sheet 114. A second adhesive layer 144 is disposed between and adhesively bonded to the inner surface of sheet 130 and the outer surface of sheet 116.

These two adhesive layers are preferably narrow elongated strips of double-sided (e.g. double-stick) adhesive tape that extend side-to-side and are attached to the sheets 114 and 116 adjacent the mouth of container 104. In this manner, the user can grasp the mouth of the outer fabric pouch, pull sheets 128 and 130 apart at the mouth and these sheets, adhesively coupled to the sheets comprising the container 104, will responsively open the mouth of container 104 to ease the insertion of ice particles into the container.

In addition, if the adhesive layers 142 and 144 extend substantially the entire width of pouch 102 or container 104, these layers will significantly reduce the possibility that ice particles will fall in between the outer fabric pouch 102 and the inner container 104.

Ice pack 100 also includes a sheet of hook fastener material 150 that is fixed to the outer surface of front sheet 128 of the outer pouch. This sheet is preferably comprised of a substrate from which a dense pattern or pile of hook-shaped protrusions extend outward. An acceptable material would be any of the wide variety of hook fastener materials sold under the trade name “Velcro®”.

Hook fastener material 150 is preferably fixed on the outer surface of outer pouch 102 in the form of an elongated strip extending laterally and horizontally across substantially the entire width of pouch 102 and adjacent to the bottom edge of pouch 102.

Hook fastener material 150 preferably has a width of between 0.25 and 1.0 inches and is preferably heat-fused to the front sheet 128. The loop material and the hook material are selected such that the four straps and the strip of hook material will form a mechanical bond sufficient to support the weight of the ice pack when filled with ice and to hold the ice pack in place when the straps are wrapped around a body part (as in FIGS. 4-7) and attached to the strip of hook material 150.

FIG. 4 shows a first method of using ice pack 100. In this arrangement, pouch 104 is placed in contact with a human arm such that the greater length of pouch 102 is parallel to the longitudinal extent of the arm. The four straps 106, 108, 110, and 112 are deflected from their as-manufactured laterally extending positions to positions in which top straps 106, 108 extend upward and straps 110 and 112 extend downward—i.e. in opposite directions.

The straps are then wrapped around the human arm until they again return to pouch 102. Since in the preferred embodiment the straps have a significant length (e.g. between 8 and 40 inches) the user may continue wrapping them around the arm a second or even a third time, thereby distributing the load of the ice pack over several circumferential wraps of the straps.

In this method for securing the ice pack, the straps 106, 110 coupled to the left side of pouch 102 and wrapping around the arm in opposite directions are then tied together at their distal ends to secure straps 106 and 110 in their wrapped position. in this manner, the two straps together can loop as many as two to six times around the user's arm. This can distribute the force holding the ice pack against the arm along the entire length of the ice pack, and not just at the ends of the ice pack.

In a similar fashion, the user can repeat the process using the two straps 108, 112 coupled to the right end of pouch 102 in the same manner to provide and additional two to six loops around the arm and over the top of the ice pack.

FIG. 5 shows a second method of using ice pack 100. In this arrangement, pouch 104 is placed in contact with a human arm such that the greater length of pouch 102 is parallel to the longitudinal extent of the arm. The four straps 106, 108, 110, and 112 are deflected from their as-manufactured laterally extending positions to positions in which top straps 106, 108 extend upward and straps 110 and 112 extend downward—i.e. in opposite directions.

The straps are then wrapped around the human arm until they again return to pouch 102. Since in the preferred embodiment the straps have a significant length (e.g. between 8 and 40 inches) the user may continue wrapping them around the arm a second or even a third time, thereby distributing the load of the ice pack over several circumferential wraps of the straps.

In this method for securing the ice pack, the straps 106, 110 coupled to the left side of pouch 102 are wrapped around the arm in opposite directions. As each wrap or loop is made around the arm the straps return to the outer pouch. As the user wraps the straps around the outer pouch 102, the user presses each strap into contact with the hook material 150. Thus each strap is secured to the hook material 150 after it makes a single wrap around the arm. However, since the straps are preferably elongate (preferably between 8 and 40 inches) the user can continue wrapping them around the arm preferably for at least one more and perhaps as many as three times. As each successive wrap of the straps passes over outer pouch 102, the strap again makes contact with the outer pouch and again makes a mechanical bond between the strap and the hook material 150.

As each successive wrap is made of straps 106 and 110, they can be made in a spiral fashion down the length of the outer pouch 102. Thus each strap can provide a plurality of individual wraps around the arm and outer pouch that are spaced apart, each being secured at a plurality of locations along the length of the strap, wherein the free portion of each strap between each successive securing location defines a separate wrap or loop around the arm. When the straps have a greater length and the body portion is smaller, as many as three wraps about the body portion can be made from a single strap.

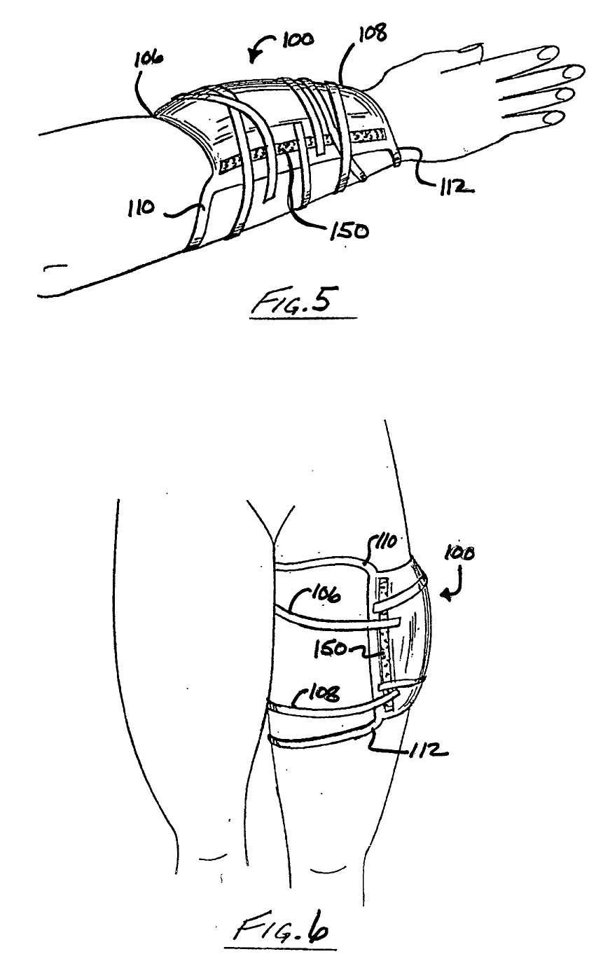

FIG. 6 shows the same method of applying the ice pack as FIG. 5, but the body portion is a leg.

FIG. 7 shows an alternative arrangement of the ice pack 100 wherein the ice pack is retained on the body portion (shown as a human torso) using a cinch that is engaged with the distal ends of the straps to hold the ice pack in position on the body portion. In the embodiment shown in FIG. 7, the outer pouch 102 with its straps 106, 108, 110 and 112 are wrapped around the torso of a human. The free distal ends of the straps are mechanically engaged to cinches 152 to hold the ends together with sufficient strength to support the ice pack on the body portion.

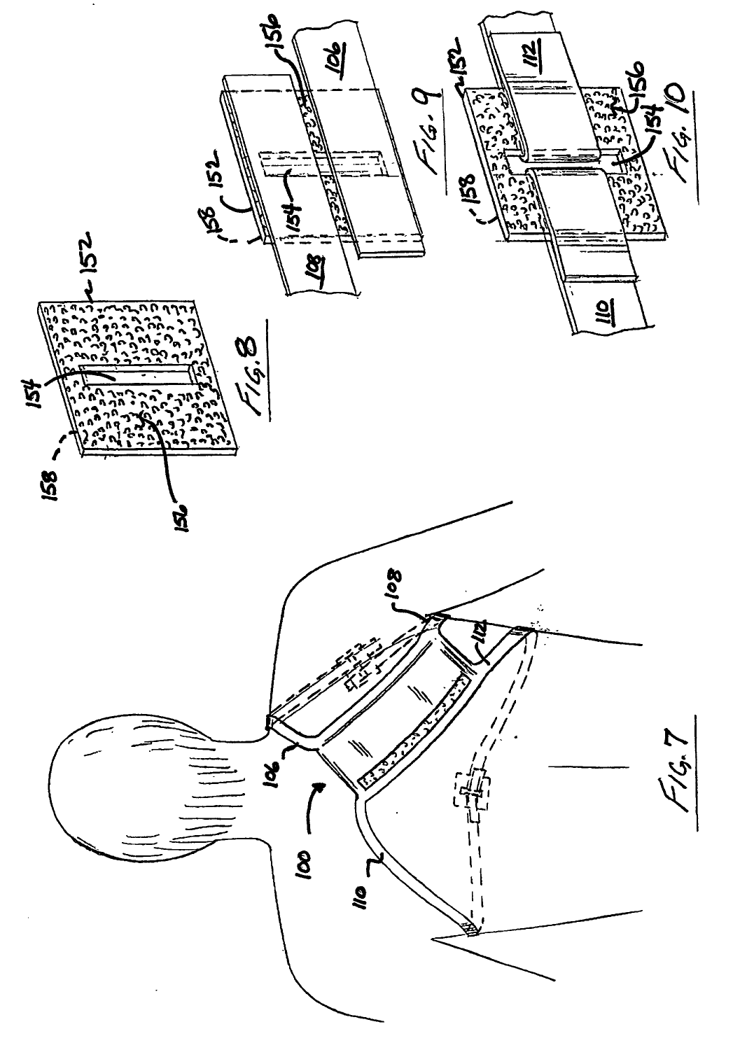

Referring now to FIGS. 8-10, cinch 152 is a planar sheet of hook material having the same characteristics as that of the strip of hook material 150. Cinch 152 is a planar sheet of hook material having an aperture 154 sized to permit one or two straps 106, 108, 110 and 112 to pass therethrough. It has two planar and opposing sides 156 (facing the viewer in FIGS. 8-10) and 158 (away from the viewer in FIGS. 10). Side 156 has the dense pattern of hooks extending therefrom that are configured to engage and mechanically lock with the loops extending from the straps. As shown in FIGS. 7 and 9, these straps can be laid side-by-side across the cinches in opposite directions but parallel to engage the hooks on the cinches. As shown in FIGS. 7 and 10, the cinches can alternatively be inserted through the aperture or slot and folded back 180 degrees. The folded-back portions of the ends of the straps can then be pressed against the hooks on side 156 to mechanically engage the hooks on side 156 and retain the ice pack in position. The advantage of the cinch arrangement of FIGS. 7 and 10 is that the folding back provides strain relief for the straps and reduces the shear stress on the hook and loop joints between the strap and the hook material.

What is claimed is:

1. An ice pack comprising:
an inner waterproof container, including:
first and second opposed planar sheets of thermoplastic film having top, bottom and two opposed side edges, the sheets of film being integrally bonded together along their bottom and side edges;
a first laterally-extending thermoplastic zipper element bonded to the top edge of the first thermoplastic sheet;
a second laterally-extending thermoplastic zipper element bonded to the top edge of the second thermoplastic sheet; wherein the first and second zipper elements flexibly and mechanically engage with one another to seal the container closed;
an outer fabric pouch enclosing the waterproof container having an inner and an outer surface and four corners, the pouch including first and second opposed planar sheets of fabric having a top edge, a bottom edge, two opposed side edges, and four comers, the first and second sheets of fabric being integrally bonded together along their bottom and side edges to define a mouth of said pouch; and
four straps having first and second ends, the first end of each of said four straps being fixed to a corresponding comer of the pouch, wherein each strap is comprised of a loop fabric having a substrate and a dense pattern of fiber loops extending from the substrate; and
at least one hook fastener sheet, the hook fastener sheet including a substrate and a dense pattern of outwardly extending hooks, wherein the hook fastener sheet is configured to mechanically and releasably engage the dense pattern of fiber loops.

2. The ice pack of claim 1, wherein the hook fastener sheet is fixed to the outer surface of the outer fabric pouch.

3. The ice pack of claim 1 wherein the hook fastener sheet is configured to be coupled to second ends of at least two of the four straps.

4. The ice pack of claim 3, wherein the hook fastener sheet defines an aperture configured to receive therethrough at least one of said four straps.

5. The ice pack of claim 4, wherein the aperture is a slot.

6. The ice pack of claim 2, further comprising first and second double-stick adhesive film respectively fixed to and between the first and second opposed planar sheets of thermoplastic film and the first and second opposed planar sheets of fabric.

7. The ice pack of claim 6, wherein substantially the entire length of the four straps are comprised of the loop fabric.

8. The ice pack of claim 7, wherein the inner waterproof container and the outer pouch are greater in width than in height and furthermore wherein the hook fastener sheet is longer than the height of the outer pouch.

9. The ice pack of claim 8, wherein a first pair of the four straps comprise an integral and elongated length of the loop fabric ultrasonically bonded to the first opposed planar sheet of fabric, and wherein a second pair of the four straps comprise an integral and elongated length of the loop fabric ultrasonically bonded to both the first and second opposed planar sheets of fabric.

10. The ice pack of claim 1, wherein the first and second opposed planar sheets of fabric are comprised of two layers, an inner non-woven layer comprising a hydrophillic material and an outer layer comprising polyester.

11. The ice pack of claim 1, wherein the four straps have a width of between 0.375 and 1.5 inches and a length of between 8 and 40 inches.

12. A method of using an ice pack comprising:
(1) an inner waterproof container of two planar sheets of thermoplastic film integrally bonded at their edges to provide a mouth having a zipper with two facing elongated and mutually inter-engaging ribs;
(2) an outer fabric pouch comprising two planar sheets of non-woven fabric, wherein the outer fabric pouch encloses the inner waterproof container;
(3) four straps fixed to and extending from the fabric pouch, wherein the four straps are comprised essentially of a loop fabric having a substrate and a dense pattern of fiber loops extending from the substrate; and
(4) a strip of hook material bonded to the outer fabric pouch, wherein the hook material comprises a substrate and a dense pattern of hooks extending therefrom, wherein the hook material and the loop fabric are inter-engageable to form a releasable mechanical bond when pressed into engagement, the method comprising the steps of:
placing the ice pack on a portion of the human body;
extending a plurality of the four straps away from the outer fabric pouch;
wrapping the plurality of the four straps around the portion of the human body; and
mechanically engaging the loop fabric of the plurality of the four straps with the hook material to retain the outer fabric pouch in contact with the portion of the human body.

13. The method of claim 12, wherein the body portion is a human leg.

14. The method of claim 13, wherein the body portion is a human arm.

15. The method of claim 14, wherein the body portion is a human torso.

16. A method of using an ice pack comprising:
(1) an inner waterproof container of two planar sheets of thermoplastic film integrally bonded at their edges to provide a mouth having a zipper with two facing elongated and mutually interengaging ribs;
(2) an outer fabric pouch comprising two planar sheets of non-woven fabric, wherein the outer fabric pouch encloses the inner waterproof container;
(3) four straps fixed to and extending from the fabric pouch, wherein the four straps are comprised essentially of a loop fabric having a substrate and a dense pattern of fiber loops extending from the substrate;
(4) a strip of hook material bonded to the outer fabric pouch, wherein the hook material comprises a substrate and a dense pattern of hooks extending therefrom, wherein the hook material and the loop fabric are inter-engageable to form a releasable mechanical bond when pressed into engagement; and
(5) a cinch of the hook material, the method comprising the steps of:
placing the ice pack on a portion of the human body;
extending at least two of the four straps away from the outer fabric pouch;
wrapping the at least two straps around the portion of the human body; and
mechanically engaging the loop fabric of the at least two straps with the cinch to retain the outer fabric pouch in contact with the portion of the human body.

17. The method of claim 16, wherein the body portion is a human leg.

18. The method of claim 17, wherein the body portion is a human arm.

19. The method of claim 18, wherein the body portion is a human torso.
